(19) United States
(12) Patent Application Publication
(10) Pub. No.: US 2022/0001115 A1
(43) Pub. Date: Jan. 6, 2022

(54) NEEDLE DEPTH SENSOR FOR INJECTOR

(71) Applicant: West Pharma. Services IL, Ltd., Ra'anana (IL)

(72) Inventors: Ran HEZKIAHU, Herzliya (IL); Ross Perry VANSTONE, Scottsdale, AZ (US); Jason W. FARRIS, Gilbert, AZ (US); Spencer PRATT, Glendale, AZ (US)

(21) Appl. No.: 17/292,579

(22) PCT Filed: Nov. 11, 2019
(86) PCT No.: PCT/US2019/060740

§ 371 Date: May 10, 2021

Related U.S. Application Data
(60) Provisional application No. 62/760,628, filed on Nov. 13, 2018.

(51) Int. Cl. A61M 5/46

(52) U.S. Cl. CPC A61M 5/46; A61M 2205/276; A61M 2205/0233; A61M 2205/13

(57) ABSTRACT

An injector includes an injector housing defining a skin contact surface and an injection needle supported within the housing and movable from a retracted position to an injection position. A circuit board is supported within the housing and movable with the injection needle, the circuit board including an electrically disconnected circuit having a contact surface. An electromechanical sensor is also supported within the housing and movable with the injection needle. The electromechanical sensor defines an electrically conductive portion that is disengaged from the contact surface of the circuit when the injection needle is in the retracted position and engaged with the contact surface of the circuit so as to electrically connect the circuit when the injection needle is in the injection position

### CROSS-REFERENCE TO RELATED APPLICATIONS

This application claims priority from U.S. Provisional Patent Application No. 62/760,628, titled “Needle Depth Sensor”, filed on Nov. 13, 2018, the entire contents of which are incorporated by reference herein.

### FIELD OF THE INVENTION

The present disclosure relates to the field of injectors, and, in particular, to an injector including a depth sensor for detecting whether a needle has reached a predetermined depth.

### BACKGROUND OF THE DISCLOSURE

Conventional injectors are configured to penetrate the skin surface of a user and dispense a dosage of a substance to the user. Generally, the substance, e.g., medicament, is desired, or required, to be delivered into a specific tissue layer underneath the skin surface for successful absorption by the body to take place. One drawback of many conventional injectors, such as auto-injectors, is that the device cannot determine, e.g., sense, whether an injection needle thereof has penetrated underneath the skin surface to at least a minimum depth required for the desired level of absorption to occur, and thus the injection to be considered successful. Accordingly, the device may inject the substance, in whole or in part, outside of the desired tissue layer and provide a false positive feedback to the user of successful delivery, which may have a harmful medical consequence.

Therefore, it would be advantageous to employ a needle depth sensor in an injector configured to monitor needle depth underneath the skin surface and only initiate, or continue, injection of the substance when the needle is positioned at a sufficient minimum depth.

### BRIEF SUMMARY OF THE DISCLOSURE

Briefly stated, one aspect of the present disclosure is directed to an injector for delivering a substance to at least a predetermined depth underneath a skin surface of a user. The injector includes an injector housing defining a skin contact surface and an injection needle supported within the housing and movable relative to the skin contact surface from a retracted position, wherein at least a tip of the injection needle is contained within the housing, to an injection position, wherein the tip of the injection needle is configured to be located at or beyond the predetermined depth underneath the skin surface of the user. A circuit board is supported within the housing and is movable with the injection needle, the circuit board including an electrically disconnected circuit having a contact surface. An electromechanical sensor is also supported within the housing and movable with the injection needle. The electromechanical sensor defines an electrically conductive portion that is disengaged from the contact surface of the circuit when the injection needle is in the retracted position and engaged with the contact surface of the circuit so as to electrically connect the circuit when the injection needle is in the injection position.

### BRIEF DESCRIPTION OF THE DRAWINGS

The following detailed description of aspects of the disclosure will be better understood when read in conjunction with the appended drawings. It should be understood, however, that the disclosure is not limited to the precise arrangements and instrumentalities shown. In the drawings:

FIG. 1 is a side elevational view of an injector in accordance with an embodiment of the present disclosure, with an injection needle of the injector in a retracted position;

FIG. 2A is a cross-sectional, elevational view of the injector of FIG. 1, taken along the sectional line 2-2 of FIG. 1, with the injection needle in the retracted position;

FIG. 2B is an enlarged perspective view of an elastically deflectable arm of the injector of FIG. 2A;

FIG. 3A is a cross-sectional, elevational view of the injector of FIG. 1, taken along the sectional line 2-2 of FIG. 1, with the injection needle in an injection position thereof;

FIG. 3B is an enlarged perspective view of the elastically deflectable arm of the injector of FIG. 3A;

FIG. 4A is a cross-sectional, elevational view of the injector of FIG. 1, taken along the sectional line 2-2 of FIG. 1, with the injection needle in a final position for injection;

FIG. 4B is an enlarged perspective view of the elastically deflectable arm of the injector of FIG. 4A;

FIG. 5 is a bottom and side perspective view of a chassis of the injector of FIG. 1 and a circuit board mounted thereto; and

FIG. 6 is schematic illustration of a circuit and controller of the injector of FIG. 1.

### DETAILED DESCRIPTION OF THE DISCLOSURE

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the injector, and designated parts thereof, in accordance with the present disclosure. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

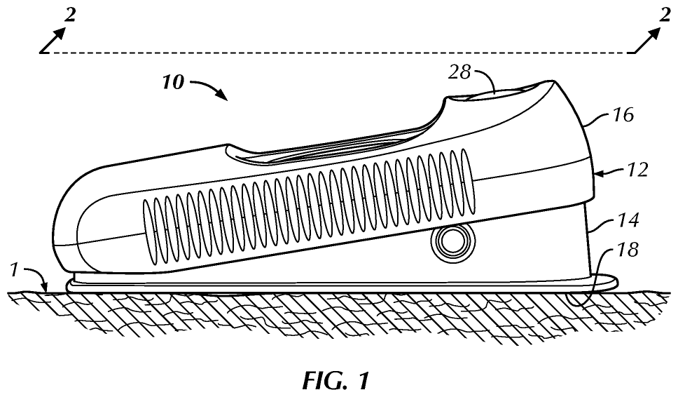

Referring to the drawings in detail, wherein like numerals indicate like elements throughout, there is shown in FIGS. 1-6 an injector, generally designated 10, in accordance with an embodiment of the present disclosure. In the illustrated embodiment, the injector 10 takes the form of a wearable injector (patch injector), such as, for example, without limitation, a wearable drug injector, but the disclosure is not so limited. The injector 10 can include an injector housing 12 having a base housing portion 14 and a cover housing portion 16 (FIG. 1) at least partially disposed over the base housing portion 14 and movably attached relative thereto. For example, the cover housing portion 16 can be pivotably attached to the base housing portion 14 such that the cover housing portion 16 is configured to pivot relative to the base housing portion 14 about an axis, though other types of relative motion are contemplated. The base housing portion 14 includes a surface 18 configured to contact a skin surface 1 of a user, e.g., a patient, the skin contact surface 18 having an opening 18a extending therethrough. In the illustrated embodiment, the skin contacting surface 18 defines a base surface of the injector housing 12, but the disclosure is not so limited. Though the injector housing 12 is described as being comprised of a base housing portion 14 and a cover housing portion 16, it is contemplated that in other embodiments the injector housing 12 can be comprised of more or less housing portions.

A chassis 22, constructed, for example, from a polymeric or metal material, combinations thereof, or the like, is mounted within the injector housing 12, i.e., between the cover housing portion 16 and the base housing portion 14, and is movably attached to the base housing portion 14. In the illustrated embodiment, the chassis 22 is pivotably attached to, and movable relative to, the base housing portion 14 proximate a rear end of the chassis 22 and the base housing portion 14, but the disclosure is not so limited. Further, the chassis 22 can be rotationally coupled to the cover housing portion 16. In the illustrated embodiment, the chassis 22 defines a cartridge slot configured, i.e., shaped and sized, to receive a substance containing cartridge 26 usable with the injector 10. Optionally, the cartridge slot cradles the cartridge 26.

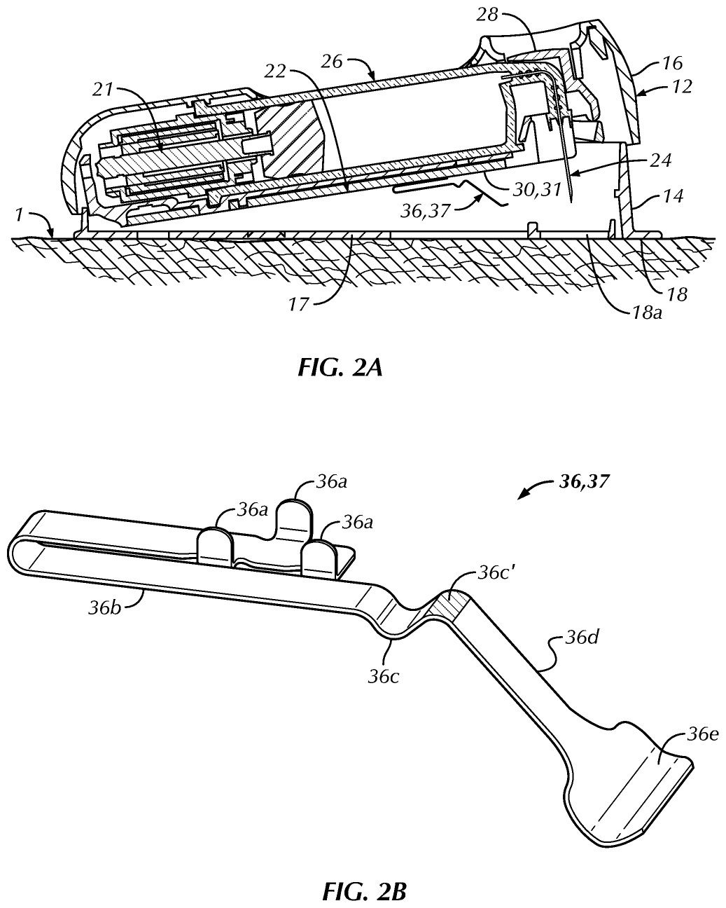

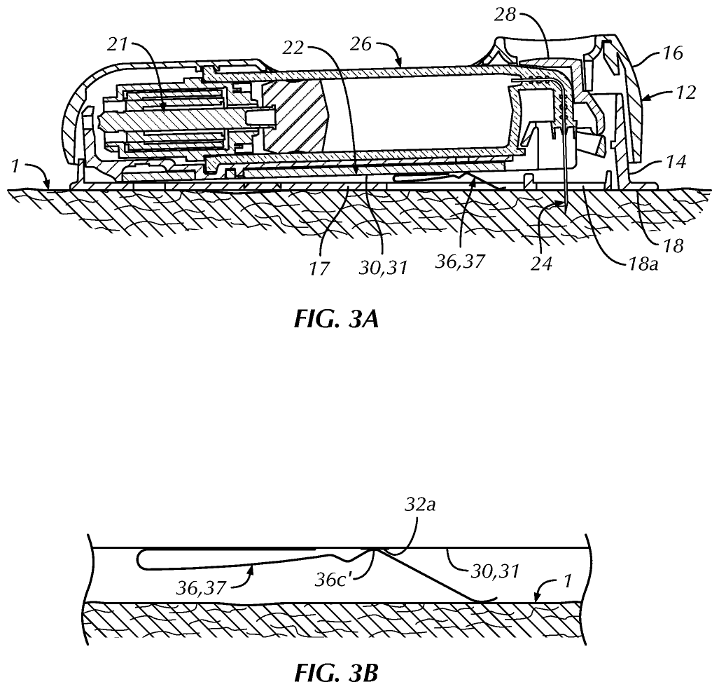

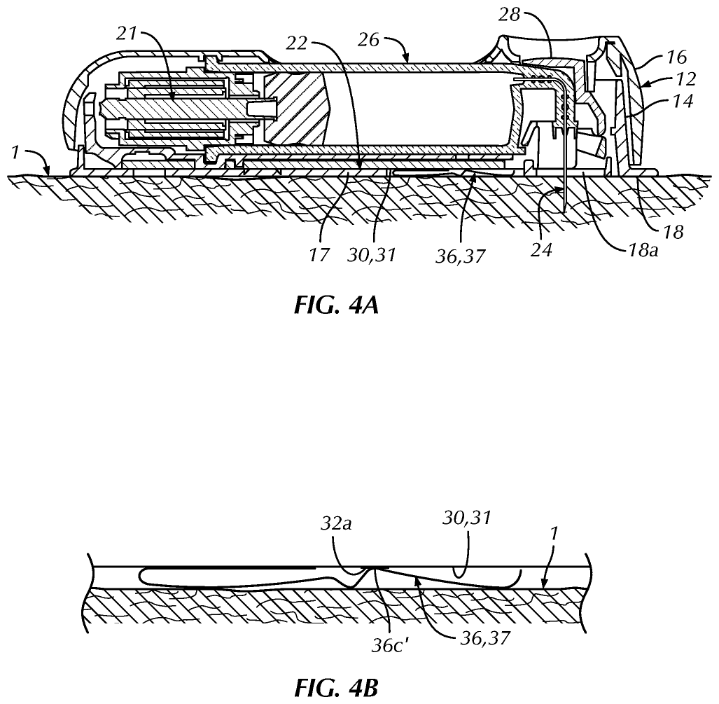

An injection needle 24 is supported within the injector housing 12 by the chassis 22. As shown in FIG. 2A, the injection needle 24 extends from a front end of the cartridge 26 and bends approximately 90° relative to a long axis of the cartridge 26, but the disclosure is not so limited. Alternatively, the injection needle 24 may be otherwise indirectly, or directly, secured to the chassis 22, or otherwise secured within the housing 12, and fluidly connectable to the cartridge 26 upon insertion of the cartridge 26 into the cartridge slot. As also should be understood, the injection needle 24 may be differently bent or may be unbent. The injection needle 24 is movable along with the chassis 22 relative to the skin contact surface 18 from a retracted position (shown best in FIG. 2A), wherein at least a tip of the injection needle 24 is contained within the base housing portion 14, to an injection position (shown best in FIG. 3A), wherein the tip of the injection needle 24 is configured to be located at least at a predetermined minimum depth underneath the skin surface 1 of the user. The injection needle 24 may also be configured to move beyond the injection position into a final position (shown best in FIG. 4A), when injection may be initiated.

The predetermined depth underneath the skin surface 1 corresponds to a minimum tissue layer underneath the skin surface 1 at which the substance within the cartridge 26 is intended or required to be delivered. For example, without limitation, some medicament may be intended or required to be delivered between at least approximately 0 mm (i.e., immediately underneath the skin surface 1 is sufficient) to approximately 12 mm underneath the skin surface 1, such as, for example, at least approximately 3.5 mm underneath the skin surface 1 for successful absorption by the body to take place. Accordingly, the predetermined minimum depth may be between approximately 0 mm to approximately 12 mm, respectively, such as, for example, approximately 3.5 mm. To reach the injection position, the injection needle 24 protrudes from the base housing portion 14 through the opening 18a in the skin contact surface 18 and penetrates through the skin surface 1 of the user to the predetermined minimum depth or beyond.

An activation button 28 is movably mounted to the injector housing 12 and is translatable/depressible (by a user) from an unactuated position (FIG. 2A) to an actuated position (FIGS. 3A, 4A). As such, the unactuated position of the activation button 28 can correspond to the retracted position of the injection needle 24, while the actuated position of the activation button 28 can correspond to the injection position of the injection needle 24, the final position of the injection needle 24, or any position of the injection needle 24 therebetween. Once the injector 10 is placed upon the skin surface 1, depression of the activation button 28 into the actuated position thereof causes (or releases) the chassis 22 (and the overlying cover housing portion 16), and, in turn, the injection needle 24, to pivot, or otherwise translate, downwardly toward the base housing portion 14, thereby advancing the tip of the injection needle 24 through the opening 18a to penetrate into the skin surface 1 of the user until the tip of the injection needle 24 reaches, or projects beyond, the predetermined minimum depth.

Turning to FIG. 5, a circuit board 30 is supported within the injector housing 12 and coupled with the injection needle 24 in order to move therewith. For example, the circuit board 30 may be mounted to the same component within the housing 12 as the injection needle 24. Also supported within the injector housing 12 and coupled with the injection needle 24 in order to move therewith is an electromechanical sensor 37. In the illustrated embodiment, the circuit board 30 takes the form of a printed circuit board (“PCB”) 31, but the disclosure is not so limited. In the illustrated embodiment, the PCB 31 is attached to an underside of the chassis 22 but may alternatively be otherwise supported by the chassis 22 or within the housing 12. In the illustrated embodiment (see, e.g., FIG. 2B), the electromechanical sensor 37 takes the form of an elastically deflectable arm 36 mounted to an underside of the PCB 31 and cantilevered therefrom, but the disclosure is not so limited. As shown, the sensor 37 extends away from the PCB 31 and toward the skin contact surface 18 of the injector 10.

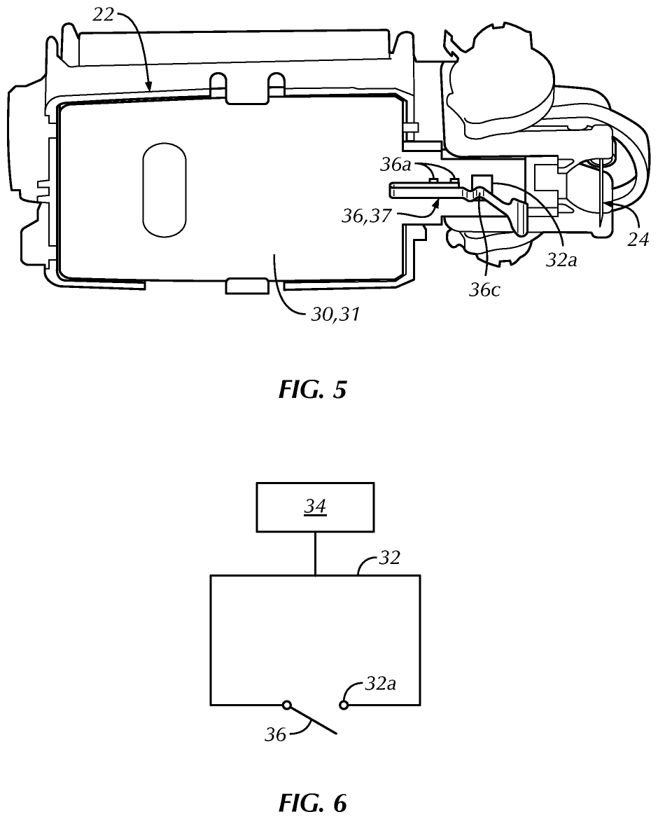

The PCB 31 includes a circuit 32 in operative communication with a controller 34 (shown schematically in FIG. 6) of the injector 10. As shown best in FIG. 6, the circuit 32 is electrically disconnected, having an open end 32a forming a contact surface. As shown best in FIG. 2A, the deflectable arm 36 is positioned in a withdrawn position within the injector housing 12 when the injection needle 24 is in the retracted position thereof. In the absence of an application of external force on the deflectable arm 36 (i.e., an unbiased orientation), such as, for example, in the withdrawn position thereof, the deflectable arm 36 is disengaged from the contact surface 32a of the circuit 32. That is, the deflectable arm 36 is disengaged from the contact surface 32a of the circuit 32 in the natural geometry of the arm 36.

In the illustrated embodiment, the deflectable arm 36 is constructed of a single monolithic component that is pre-shaped to a specific geometry, but the disclosure is not so limited. For example, it is contemplated that the deflectable arm 36 may be constructed of multiple integral components. As shown best in FIGS. 2B, 3B, and 4B, the deflectable arm 36 is mounted to the circuit 32 via legs 36a. In the illustrated embodiment, the deflectable arm 36 includes three legs 36a, but the disclosure is not so limited. In the illustrated embodiment, the deflectable arm 36 also includes a generally horizontally oriented, i.e., oriented generally parallel with the PCB 31, U-shaped portion 36b extending from the mounting legs 36a, a generally sinusoidally shaped portion 36c extending generally horizontally from the U-shaped portion 36b, and a generally linear arm 36d angling toward the skin contact surface 18 from the portion 36c. A terminal, free end 36e of the portion 36d takes the form of a curved and widened portion 36e configured to slide along the skin surface 1 of the user upon contact therewith, as will be described in further detail below. As shown best in FIG. 5, at least a portion of the generally sinusoidally shaped portion 36c is generally axially aligned with the contact surface 32a of the circuit 32. At least a portion of the deflectable arm 36 is constructed of an electrically conductive material, but the disclosure is not so limited. In the illustrated embodiment, for example, without limitation, a portion 36c′ of the generally sinusoidally shaped portion 36c may be constructed of an electrically conductive material. The deflectable arm 36 may be constructed in whole or in part of a metal or polymeric material, a combination thereof, or the like, having the material properties to perform the functions of the arm 36 described herein, such as, for example, elastic deflection and electrical conductivity.

Additionally, or alternatively, at least a portion of the deflectable arm 36 may be provided with an electrically conductive plating/coating.

The U-shaped portion 36b contributes to the elastic deflectability of the cantilevered arm 36. Namely, application of an external force having at least an axial component (relative to the U-shaped portion 36b) on the arm 36 deflects the arm 36 about an axis of symmetry of the U-shaped portion 36b (as shown between FIGS. 2B and 3B, 4B), thereby elastically deflecting the generally sinusoidally shaped portion 36c toward the contact surface 32a of the circuit 32. Application of a sufficient external axial force onto the deflectable arm 36 (i.e., a biased orientation) deflects the sinusoidally shaped portion 36c, thereby engaging at least the electrically conductive portion 36c′ into engagement with the contact surface 32a of the circuit 32 (as shown between FIGS. 2B and 3B), thereby electrically connecting the circuit 32, as will be described in further detail below. For example, without limitation, an external force within the range of approximately 0.02 Newton and approximately 8 Newtons, may be sufficient to elastically deflect the arm 36 into engagement with the contact surface 32a.

In use, the injector 10 is placed on the skin surface 1 of the user, with the injection needle in the retracted position and the elastically deflectable arm 36 in the withdrawn position. In some embodiments, the injector 10 may include a safety latch 17 pivotably attached to the base housing portion 14, and movable between a first position (FIGS. 1, 2A, 3A, 4A), wherein the safety latch 17 extends generally flush with the skin contacting surface 18 of the injector 10, and a second position (not shown), wherein the safety latch 17 is pivoted away, i.e., downwardly, from the skin contacting surface 18. The safety latch 17 is moved into the first position when the injector 10 is placed on the skin surface 1.

The user then depresses the activation button 28 to initiate movement of the chassis 22 toward the skin contacting surface 18, and, in turn move the injection needle 24 toward the injection position thereof. Movement of the chassis 22 toward the skin contacting surface 18 also moves the elastically deflectable arm 36 toward an advanced position thereof (FIG. 3A), wherein at least a portion thereof, e.g., the terminal portion 36e, projects beyond the skin contact surface 18 of the injector 10 via the opening 18a and the curved and widened terminal free end 36e of the arm 36 advances toward the skin surface 1 of the user. Optionally, where a safety latch 17 is employed, the elastically deflectable arm 18 may move from the withdrawn position thereof into the advanced position thereof through a slot (not shown) in the safety latch 17 but may otherwise advance toward the skin surface 1 via another path.

In some embodiments, such as shown in FIG. 3A, the elastically deflectable arm 36 is dimensioned to contact the skin surface 1 upon penetration of the injection needle 24 to a predetermined depth underneath the skin surface 1, e.g., approximately 1-2 mm. Alternatively, the elastically deflectable arm 36 may dimensioned to contact the skin surface 1 generally simultaneously with contact of the injection needle 24 with the skin surface 1. Upon contact of the elastically deflectable arm 36 with the skin surface 1, and also as the injection needle 24 continues to project deeper underneath the skin surface 1 toward the injection position thereof, the skin surface 1 applies an external axial force onto the arm 36, elastically deflecting the arm 36 toward the contact surface 32a of the circuit 32, e.g., about an axis of symmetry of the U-shaped portion 36b. As shown in FIG. 3A, the arm 36 is configured to engage the contact surface 32a of the circuit 32 when the injection needle 24 reaches the injection position thereof, i.e., at the predetermined minimum depth of the injection needle 24 underneath the skin surface 1, thereby closing and electrically connecting the circuit 32. For example, the arm 36 may be configured (i.e., shaped and dimensioned) to be biased/deflected by the skin surface 1 into engagement with the contact surface 32a once the injection needle 24 has penetrated 3.5 mm underneath the skin surface 1.

As shown in FIG. 4A, the chassis 22 may continue to advance the injection needle 24 to penetrate further/deeper underneath the skin surface 1 beyond the minimum depth until a final position thereof for successful injection of the substance within the cartridge 26. The arm 36 remains in contact with the contact surface 32a during such further penetration of the injection needle 24, maintaining electrical connection of the circuit 32. Continued application of the external force onto the arm 36, and particularly onto the terminal free end 36e, angularly flexes the generally linear arm portion 36d along the skin surface 1 (FIG. 4B). That is, the crests and troughs of the generally sinusoidal portion 36c of the elastically deflectable arm 36 permit obtuse angular flexing of the generally linear arm portion 36d along the skin surface 1 toward an orientation generally parallel with the skin surface 1. The curved and widened portion 36e at the terminal end of the generally linear arm portion 36d permits generally smooth sliding of the generally linear arm portion 36d along the skin surface during the obtuse angular deflection thereof.

The circuit is operatively connected to the controller to provide feedback to the controller 34 (in a manner well understood by those of ordinary skill in the art) and the controller 34 is configured to account for the connection state of the circuit 32 in decision-making logic thereof. In one configuration, for example, the electrical circuit 32 is binary, i.e., has either an electrically connected or disconnected state. In the connected state, the circuit 32 may transmit a signal to the controller 34, whereas in the disconnected state, the circuit 32 may provide no signal to the controller 34. Advantageously, electric connection of the circuit 32 indicates that the injection needle 24 is positioned adequately, i.e., deep enough, underneath the skin surface 1 to enable successful injection of the substance within the cartridge 26 that will be properly absorbed by the body. Conversely, electric disconnection of the circuit 32 indicates that the injection needle 24 is not positioned adequately to enable a successful injection of the substance within the cartridge 26. The controller 34 is, therefore, configured to prevent operation of the injector 10, e.g., prevent or pause injection of the substance within the cartridge 26, when the circuit 32 is electrically disconnected. For example, without limitation, the controller 34 may be configured to pause or stop the driving mechanism 21 (see FIGS. 2A, 3A, 4A) responsible for ejecting the substance from the cartridge 26. Optionally, the controller 34 may, for example, also be configured to output an error message, e.g., an audible and/or visual error message. Closure of the circuit 32 indicates that the injection needle 26 is at least a desired depth underneath the skin surface 1. Upon receiving feedback that the circuit 32 is electrically connected, the controller 34 is configured to permit, initiate or resume operation of the injector 10, e.g., initiate of resume injection of the substance within the cartridge 26 via the injection needle 24.

Further advantageously, because the arm 36 is elastically deflectable, the elastically deflectable arm 36 is configured to return toward the natural geometry thereof, away from the contact surface 32a of the circuit 32, upon reduction of the external force applied thereon by the skin surface 1. Accordingly, movement of the injection needle 24, e.g., due to movement between the injector 10 and the skin surface 1, that results in the injection needle 24 no longer penetrating the skin surface 1 at, or beyond, the minimum depth, corresponds to the external force applied by the skin surface 1 onto the arm 36 no longer being sufficient to bias the arm 36 into engagement with the contact surface 32a. Accordingly, the arm 36 disengages from the contact surface 32a and electrically disconnects the circuit 32. The controller 34, therefore, takes appropriate action as previously described. Therefore, the arm 36 provides dynamic, generally real-time feedback of relative movement between the injector 10 and the skin surface 1, which correlates to the injection needle 24 depth underneath the skin surface 1.

Other advantages of the elastically deflectable arm 36 include the absence of a hysteresis effect. Moreover, the user need not perform any calibration of the arm 36 prior to use. Positioning of the arm 36 within the injector housing 12 prior to use of the injector 10 reduces any likelihood of damage thereto. Yet further, the elasticity of deflection of the arm 36 permits the continuous use of the elastically deflectable arm 36, e.g., during the entirety of the injection, to monitor needle depth.

It will be appreciated by those skilled in the art that changes could be made to the embodiments described above without departing from the broad inventive concept thereof. It is understood, therefore, that this invention is not limited to the particular embodiment(s) disclosed, but it is intended to cover modifications within the spirit and scope of the present disclosure, as set forth in the appended claims.

1. An injector for delivering a substance to at least a predetermined depth underneath a skin surface of a user, the injector comprising:
an injector housing defining a skin contact surface;
an injection needle supported within the housing and movable relative to the skin contact surface from a retracted position, wherein at least a tip of the injection needle is contained within the housing, to an injection position, wherein the tip of the injection needle is configured to be located at or beyond the predetermined depth underneath the skin surface of the user;
a circuit board supported within the housing and movable with the injection needle, the circuit board including an electrically disconnected circuit having a contact surface;
an electromechanical sensor supported within the housing and movable with the injection needle, the electromechanical sensor defining an electrically conductive portion that is disengaged from the contact surface of the circuit when the injection needle is in the retracted position and engaged with the contact surface of the circuit so as to electrically connect the circuit when the injection needle is in the injection position.

2. The injector of claim 1, wherein the electromechanical sensor is in an unbiased orientation when the electrically conductive portion is disengaged from the contact surface and the electromechanical sensor is in a biased orientation when the electrically conductive portion thereof is engaged with the contact surface.

3. The injector of claim 1, wherein the electromechanical sensor is positioned in a withdrawn position within the housing when the injection needle is in the retracted position, and the electromechanical sensor is positioned in an advanced position, wherein at least a portion of the electromechanical sensor extends beyond the skin contact surface and is engaged with the skin surface of the user, when the injection needle is in the injection position.

4. The injector of claim 1, wherein the electrically conductive portion of the electromechanical sensor is configured to disengage from the contact surface of the circuit and electrically disconnect the circuit upon movement of the injection needle from the injection position toward the retracted position.

5. The injector of claim 1, further comprising a controller in operative communication with the circuit, the controller being configured to prevent operation of the injector when the circuit is electrically disconnected, and the controller being configured to initiate or resume operation of the injector when the circuit is electrically connected.

6. The injector of claim 1, wherein the injector housing includes a base housing portion defining the skin contact surface and a chassis movable relative to the base housing portion.

7. The injector of claim 6, wherein the chassis is pivotably attached to the base housing portion.

8. The injector of claim 6, wherein the injection needle, the circuit board, and the electromechanical sensor are supported by the chassis.

9. The injector of claim 6, wherein the electromechanical sensor comprises an elastically deflectable arm configured to deflect out of a natural geometry thereof and toward the contact surface of the circuit upon application of an external force thereon, and configured to return toward the natural geometry thereof, away from the contact surface of the circuit, upon reduction of the external force thereon.

10. The injector of claim 9, wherein the elastically deflectable arm is cantilevered from the circuit board.

11. The injector of claim 10, wherein the elastically deflectable cantilevered arm comprises a free terminal end configured to slide along the skin surface of the user upon contact therewith in addition to deflecting toward the contact surface of the circuit due to the external force applied by the contact with the skin surface.

12. The injector of claim 11, wherein the elastically deflectable arm includes a generally horizontally oriented U-shaped portion opposite the free terminal end thereof, the horizontally oriented U-shaped portion configured to enable the elastic deflection toward the contact surface.

13. The injector of claim 12, wherein the free terminal end comprises a curved and widened portion to slide along the skin surface.

14. The injector of claim 12, wherein the predetermined depth underneath the skin surface of the user is approximately 3.5 mm.

15. The injector of claim 12, wherein the circuit board is a PCB.
